(19) United States
(12) Patent Application Publication
(10) Pub. No.: US 2026/0294681 A1
(43) Pub. Date: Oct. 1, 2026

(54) DIATHERMY PROBES FOR OPHTHALMIC PROCEDURES

(71) Applicant: Alcon Inc., Fribourg (CH)

(72) Inventors: Nils Benjamin Heinemann, Degersheim (CH); Simon Hildebrand, Kreuzlingen (CH); Simon Nicola Kunz, Zurich (CH)

(21) Appl. No.: 19/563,188

(22) Filed: Mar. 11, 2026

Related U.S. Application Data
(60) Provisional application No. 63/780,646, filed on Mar. 31, 2025.

(51) Int. Cl. A61F 9/007; A61B 18/14

(52) U.S. Cl. CPC A61F 9/007; A61B 18/1477; A61B 2018/1425

(57) ABSTRACT

In certain embodiments, a diathermy probe is provided that includes a handpiece and an electrode assembly extending distally from the handpiece. The electrode assembly includes an outer electrode and an inner electrode disposed within and extending through the outer electrode, where the inner electrode extends further distally from the handpiece than the outer electrode. In certain embodiments, the outer electrode and the inner electrode share a common longitudinal axis along an entire length of each of the outer electrode and the inner electrode.

### INTRODUCTION

Microsurgical procedures frequently involve precision sealing, cutting, and/or removing of various body tissues. For example, certain ophthalmic surgical procedures may involve sealing, cutting, and/or removing tissues within the posterior segment of the eye. During such procedures, a diathermy probe may be used to control unwanted bleeding in the eye, repair retinal detachments, remove abnormal tissues, etc. When using the diathermy probe, a distal tip is inserted into the eye and held in close proximity to target tissue to operate thereon. Therefore, use of the diathermy probe requires great care and accuracy to ensure only targeted tissue is operated upon.

Typically, bipolar diathermy probes include one electrode disposed around, or at least near, another electrode at the distal tip. However, because the two electrodes are in close proximity to each other at the distal tip, a portion of one electrode may block the surgeon’s view of the target tissue and/or surrounding tissue in the eye. Consequently, the surgeon may struggle to adequately see the target tissue, which can make it difficult to operate thereon and potentially lead to unwanted and unintentional trauma to ocular tissues. The visual restrictions may therefore limit the efficiency of bipolar diathermy probe-involved operations and may increase the amount of time needed to complete ophthalmic procedures, which further increases patient risk.

Conventional bipolar diathermy probes also include a connection assembly at a proximal end. At the connection assembly, the electrodes are conductively coupled to sheet metal pins that relay charges (or electrical current) from an electrical source to the electrodes. The connection assembly often includes multiple conductive connections, bent electrodes, and/or additional bridging wire(s). Consequently, a large handpiece may be needed to house such assemblies, which may make holding the diathermy probe uncomfortable for the surgeon due to the handpiece’s size and/or weight. In addition, more time and/or parts may be needed to manufacture such assemblies. These assembly requirements may therefore increase manufacturing costs while also limiting the manufacturing efficiency of bipolar diathermy probes.

### BRIEF SUMMARY

The present disclosure relates generally to ophthalmic surgical instruments, such as diathermy probes, for performing ophthalmic surgical procedures.

In certain embodiments, a diathermy probe is provided. The diathermy probe includes a handpiece and an electrode assembly extending distally from the handpiece. The electrode assembly includes an outer electrode and an inner electrode disposed within and extending through the outer electrode, where the inner electrode extends further distally from the handpiece than the outer electrode.

In certain embodiments, another diathermy probe is provided. The other diathermy probe includes a handpiece and an electrode assembly extending distally from the handpiece. The electrode assembly includes an outer electrode and an inner electrode disposed within and extending through the outer electrode, where the outer electrode and the inner electrode share a common longitudinal axis along an entire length of each of the outer electrode and the inner electrode. The diathermy probe further includes a first sheet metal pin conductively coupled to the outer electrode by a first adhesive connection at a proximal end of the handpiece, and a second sheet metal pin conductively coupled to the inner electrode by a second adhesive connection at the proximal end of the handpiece.

In certain embodiments, yet another diathermy probe is provided. The other diathermy probe includes a handpiece and an electrode assembly extending distally from the handpiece. The electrode assembly includes an outer electrode and an inner electrode disposed within and extending through the outer electrode, where the outer electrode and the inner electrode share a common longitudinal axis along an entire length of each of the outer electrode and the inner electrode. The diathermy probe further includes a first sheet metal pin conductively coupled to the outer electrode by a first crimp connection at a proximal end of the handpiece, and a second sheet metal pin conductively coupled to the inner electrode by a second crimp connection at the proximal end of the handpiece.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows an example ophthalmic surgical system that may be used to perform ophthalmic procedures on an eye, according to certain embodiments.

FIG. 1B shows example components of a surgical console of the ophthalmic surgical system shown in FIG. 1A, according to certain embodiments.

FIG. 2A is a side view of an example probe of the ophthalmic surgical system of FIGS. 1A-1B, according to certain embodiments.

FIG. 2B illustrates a distal tip configuration of the probe shown in FIG. 2A, according to certain embodiments.

FIG. 3A illustrates a cross-sectional side view of an eye with the probe of FIGS. 2A-2B inserted therein, according to certain embodiments.

FIG. 3B illustrates an isometric view of a distal tip of the probe of FIGS. 2A-2B at a treatment area of the eye of FIG. 3A.

FIGS. 4A-4B illustrate cross-sectional views of an example connection assembly that can be used with the probe shown in FIGS. 2A-2B, according to certain embodiments.

FIGS. 5A-5B illustrate cross-sectional views of another example connection assembly that can be used with the probe shown in FIGS. 2A-2B, according to certain embodiments.

FIGS. 6A-6B show side views of another example connection assembly that can be used with the probe shown in FIGS. 2A-2B, according to certain embodiments.

FIG. 6C shows an isometric perspective view of sheet metal pins shown FIGS. 6A-6B, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

During ophthalmic surgeries, diathermy probes are often used to apply localized heat at target tissue within an eye. To generate the localized heat, a charge is alternatively applied to two electrodes of a diathermy probe (e.g., a bipolar diathermy probe), which thereby creates a high-frequency electrical current that acts as a heat source. The heat generated by the diathermy probe may be used to, for example, coagulate tissue to stop bleeding, seal retinal tears or holes, remove abnormal tissues (e.g., tumors or proliferative membranes), bridge blood vessels, mark holes, etc. Additionally, diathermy probes may be used in retinotomy, e.g., for proliferative vitreoretinopathy (PVR) amotio or to create retinal breaks, which involve cutting into the retina to drain fluid or gain access to the subretinal space. Therefore, use of the diathermy probe requires great precision to ensure heat is only applied to the target tissue, so that damage does not occur to other surrounding tissues.

Current bipolar diathermy probes often include one electrode disposed within, or at least near, another electrode. For example, one electrode may be disposed within another electrode, and a distal end of each of the electrodes may be adjacent to, or substantially near each other at a distal tip of the diathermy probe. As such, both of the electrodes may occupy a certain amount of spatial volume at a distal tip of the diathermy probe. However, by having both electrodes completely (or near completely) extend to the distal tip, one or both of the electrodes may hinder the surgeon’s view of the target tissue and/or surrounding tissue. When the surgeon cannot adequately see the target tissue, it increases the likelihood of unwanted and unintentional trauma to ocular tissues, and may also increase a duration of the surgery as the surgeon needs to take extra care in order to avoid unwanted trauma, which further increases risk to the patient.

Accordingly, the embodiments described herein provide diathermy probes that include an inner electrode extending further distally from a handpiece than an outer electrode. For example, the diathermy probes herein separate a distal end of the inner electrode from a distal end of the outer electrode at the distal tip, such that the outer electrode occupies less spatial volume at the distal tip, thereby affording the surgeon greater visibility of target tissue and/or surrounding tissue within the eye. The diathermy probes described herein, therefore, provide surgeons with an improved view of the treatment area within the eye, which thereby facilitates safer, quicker, and more efficient ophthalmic procedures.

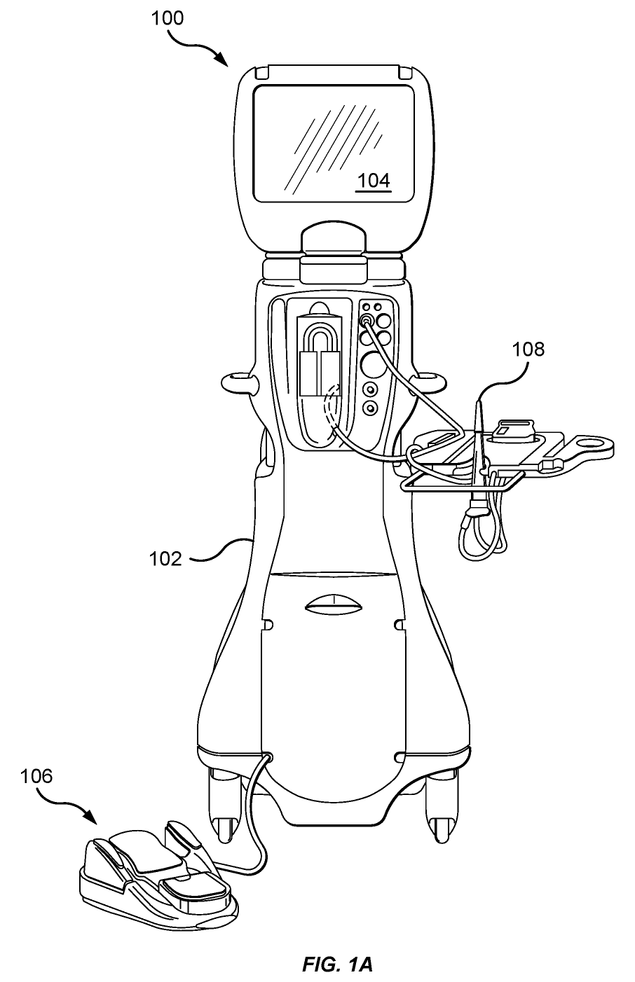

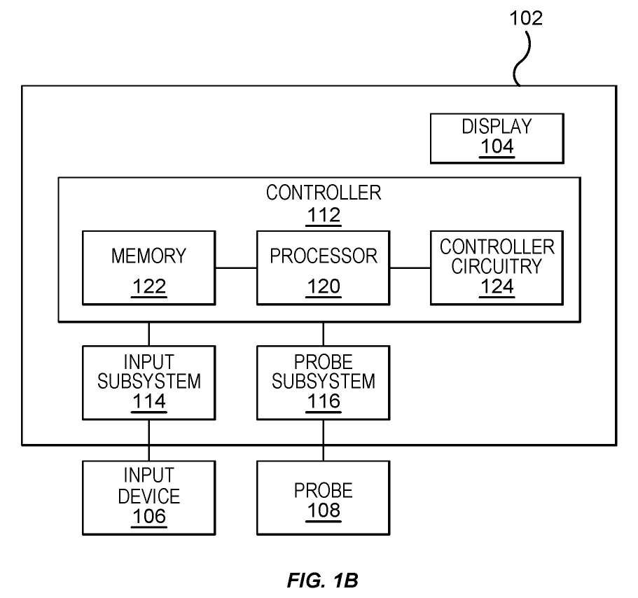

FIG. 1A shows an example ophthalmic surgical system 100 that may be used to perform ophthalmic procedures on an eye, according to certain embodiments. The ophthalmic surgical system 100 includes a console 102 (also referred to as a “surgical console”), which includes a display 104, an input device 106 (e.g., a foot pedal), and a probe 108 (also referred to as an “instrument” or an “ophthalmic surgical instrument”). The components of the ophthalmic surgical system 100 and the surgical console 102 are mechanically and/or electrically coupled as shown and described in more detail with reference to FIG. 1B.

FIG. 1B shows example components of the surgical console 102 of the ophthalmic surgical system 100 shown in FIG. 1A, according to certain embodiments. As shown, the surgical console 102 includes a controller 112, an input subsystem 114, a probe subsystem 116, and a display 104. The controller 112 controls the operation of the surgical console 102 and is illustrated as being operationally coupled by a wired or wireless connection to the input device 106 via input subsystem 114, and to the probe 108 via the probe subsystem 116. The controller 112 includes a processor 120, a memory 122, and controller circuitry 124.

The processor 120 may be any type of general purpose processor or could be a processor specifically designed for driving the subsystems illustrated in FIG. 1B, such as an application-specific integrated circuit (“ASIC”). The processor 120 may be, or include, a microprocessor, a microcontroller, an embedded microcontroller, a programmable digital signal processor, or any other programmable device operable to execute instructions stored in the memory 122 for operating the surgical console 102. For example, the processor 120 may execute instructions in the memory 122 to receive inputs provided by the input device 106 through the input subsystem 114 and, in response, send instructions to the probe subsystem 116 for operating the probe 108. Further, the processor 120 may execute instructions to generate user interface view for display by the display 104. In some instances, the processor 120 may also be or include a programmable gate array, programmable array logic, or any other device of combinations of devices operable to process electric signals.

The memory 122 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 122 stores instructions executed by the processor 120. In example embodiments, functionality disclosed herein can be provided by the processor 120 and the memory 122 (i.e., software based), by the controller circuitry 124 (i.e., hardware based), or by a combination thereof. The memory 122 may include code stored thereon. The code may include instructions that may be executable by the processor 120. The code may be created, for example, using any programming language, including but not limited to, C, C++, Java, Python, Rust, or any other programming language (including assembly languages, hardware description languages, and database programming languages). In some instances, the code may be a program that, when executed by the processor 120, causes the surgical console 102 to operate subsystems 114 and/or 116 for, e.g., driving the probe 108 or other devices in communication with the surgical console 102.

The probe 108 may be any suitable ophthalmic surgical instrument that can be operated on the basis of the embodiments described herein. For example the probe 108 may be a diathermy probe (also referred to as a “diathermy handpiece” or a “diathermy needle”).

The probe subsystem 116 is configured to facilitate the operation of the probe 108. For example, the probe subsystem 116 may control the operations (e.g., activation and deactivation) of a component of the probe 108.

The input device 106 may be any device that is capable of receiving commands from the user of the surgical console 102 in order to operate the probe 108 and/or other components of the surgical console 102. In FIG. 1A, the input device 106 is illustrated as a foot pedal, however, other types of input devices are also within the scope of the disclosure. In one example, the user provides a command to the input device 106, which is received and relayed to the controller 112 by the input subsystem 114. In response, the controller 112 sends instructions to the probe subsystem 116 to control the operations of the probe 108 based on the user command.

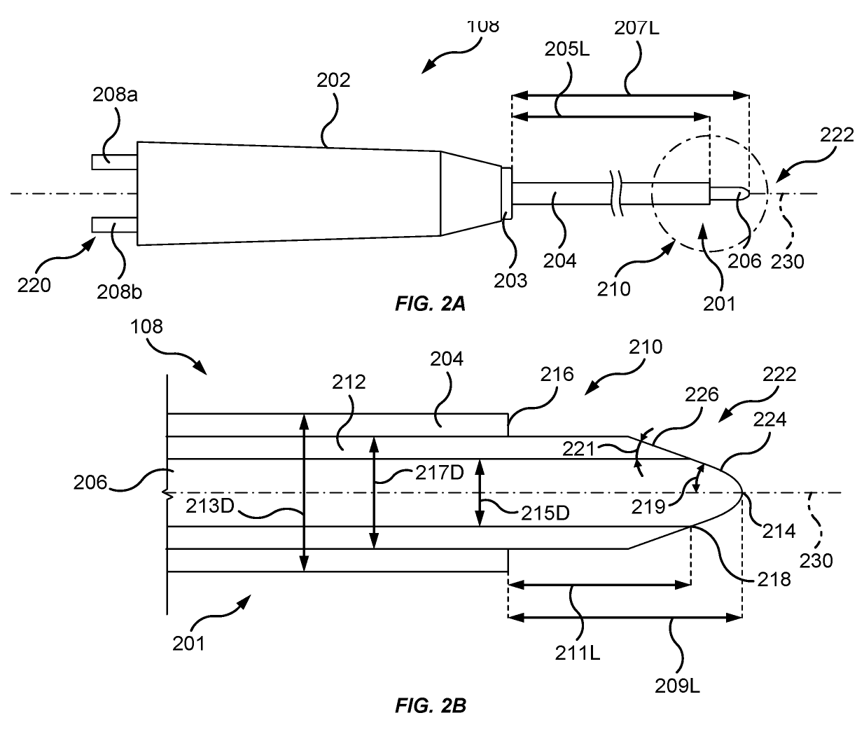

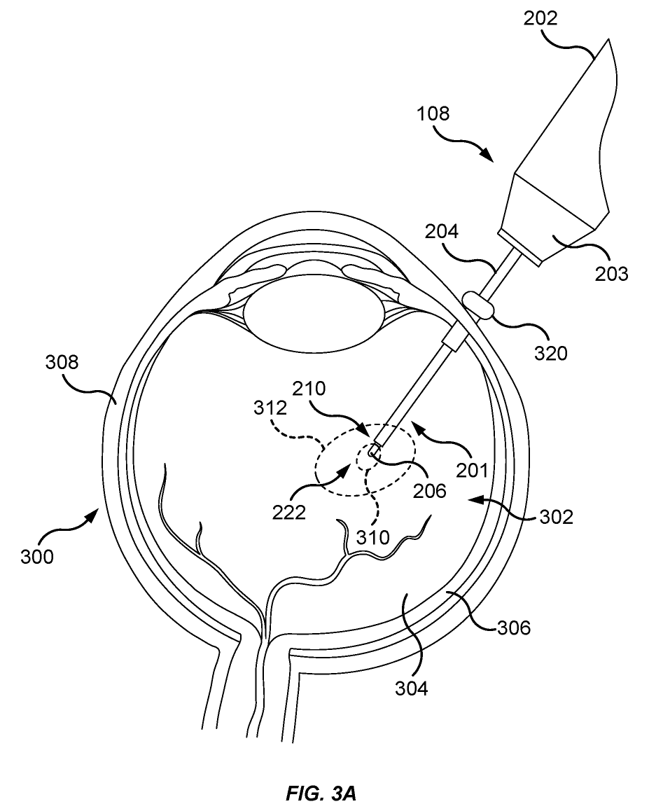

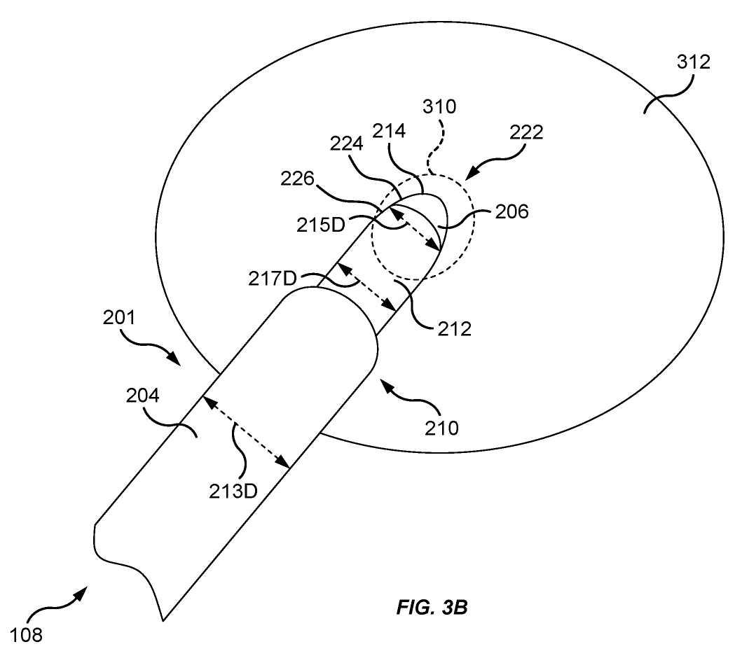

FIG. 2A is a side view of the probe 108 of the ophthalmic surgical system 100 of FIGS. 1A-1B, according to certain embodiments. The probe 108 is a diathermy probe (sometimes referred to herein as a “diathermy handpiece” or a “diathermy needle”) which may be used, for example, to generate and apply heat to target tissue inside an eye as described in further detail with reference to FIGS. 3A-3B. The surgical console 102 and/or the controller 112 are configured to control the probe 108 and operational features thereof.

The probe 108 includes a handpiece 202 and a base 203. In certain embodiments, the handpiece 202 is configured to be held by a user, such as a surgeon. For example, the handpiece 202 may be ergonomically contoured to substantially fit the hand of the user. In certain embodiments, the outer surface may be textured or have one or more gripping features formed thereon, such as one or more grooves and/or ridges. The handpiece 202 may be made from any materials commonly used for such instruments and suitable for ophthalmic surgery. For example, the handpiece 202 may be formed of a lightweight aluminum, a polymer, or other suitable material. In some embodiments, handpiece 202 may be sterilized and used in more than one surgical procedure, or may be a single-use device.

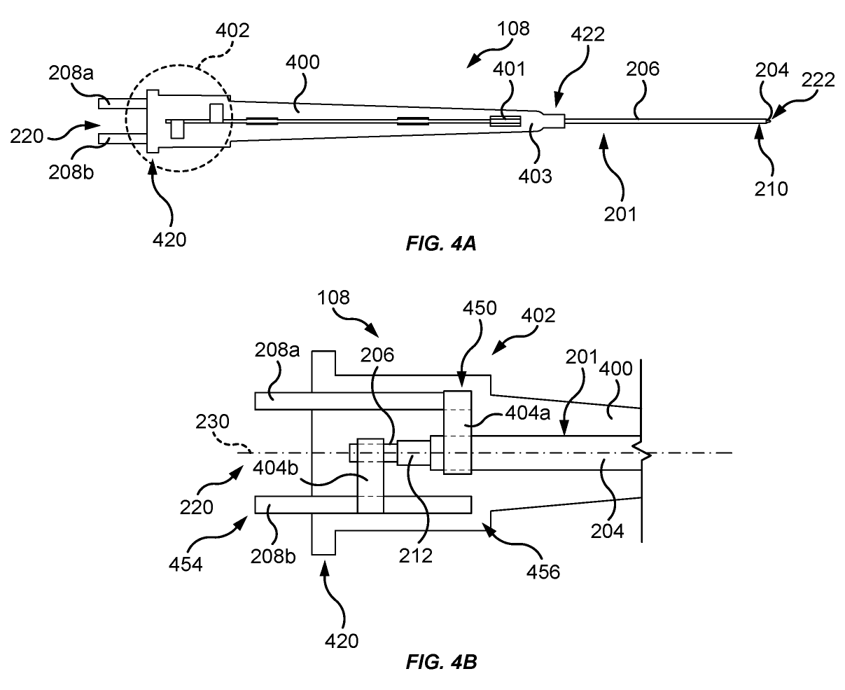

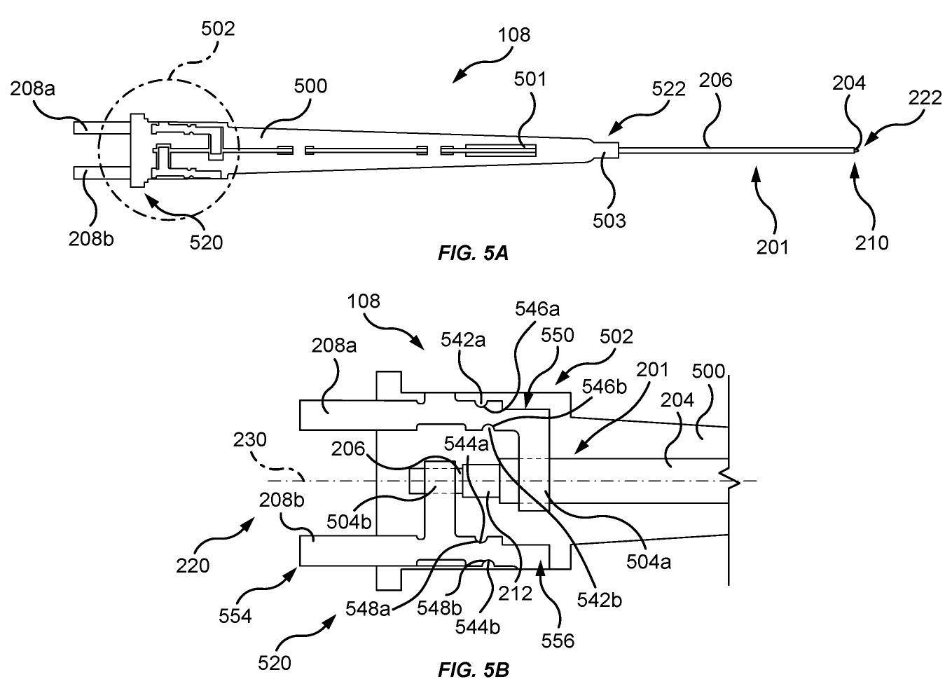

The handpiece further includes a first sheet metal pin 208a and a second sheet metal pin 208b (collectively referred to as “sheet metal pins 208a-b”) at a proximal end 220. The sheet metal pins 208a-b provide an electrical connection for charges (or electrical currents) to be routed into an interior lumen of the probe 108 (shown in FIGS. 4A-4B and 5A-5B). For example, the sheet metal pins 208a-b may provide a connection between the probe 108 and an electrical source within the surgical console 102.

The probe 108 further includes an electrode assembly 201 extending distally from the handpiece 202. The electrode assembly 201 may be inserted into an eye, e.g., through a trocar cannula, to coagulate tissues within the eye. The electrode assembly 201 comprises an outer electrode 204 and an inner electrode 206 disposed within and extending through the outer electrode 204. The outer electrode 204 and the inner electrode 206 define a major (longitudinal) axis 230 of the probe 108. Additionally, the outer electrode 204 and the inner electrode 206 share a common longitudinal axis, i.e., the longitudinal axis 230, along an entire length of each of the outer electrode 204 and the inner electrode 206, which is shown in further detail with reference to FIGS. 4A and 5A.

At a distal tip 210 (or a distal end 222) of the probe 108, the inner electrode 206 is offset from the outer electrode 204. In other words, the inner electrode 206 extends further distally from the base 203 of the handpiece 202 than the outer electrode 204. For example, the outer electrode 204 extends from the base 203 by a first length 205L, and the inner electrode 206 extends from the base 203 by a second length 207L that is greater than the first length 205L. As an example, the second length 207L is greater than the first length 205L by 0.5 millimeters (mm) to 2 mm.

The first length 205L may be between 20.5 mm and 39.5 mm (e.g., between 21 mm and 39 mm, 21.5 mm and 38.5 mm, 22 mm and 38 mm, or 22.5 mm and 37.5 mm), and the second length 207L may be between 20 mm and 40 mm (e.g., between 20.5 mm and 39.5 mm, 21 mm and 39 mm, 22.5 mm and 38.5 mm, or 23 mm and 38 mm). In certain embodiments, the first length 205L and/or the second length 207L may further vary based on the size of a patient’s eye.

For clarity, an enlarged cross-sectional view of the distal tip 210 is illustrated in FIG. 2B, which is described in further detail below. A proximal end of the electrode assembly 201 may be directly or indirectly attached to the sheet metal pins 208a-b within an interior lumen of handpiece 202, e.g., as shown in FIGS. 4A-4B and 5A-5B.

At the distal tip 210 of the probe 108, the electrode assembly 201 is configured to apply heat to a target tissue within a patient’s eye. To generate the heat, a charge is alternatively applied to the outer electrode 204 and the inner electrode 206, which thereby creates a high-frequency electrical current that acts as a heat source at the distal tip 210. The generated heat may then be applied to the target tissue to, for example, coagulate tissue to stop bleeding, seal retinal tears or holes, remove abnormal tissues (e.g., tumors or proliferative membranes), mark tears or holes, etc. Applying the heat to the target tissue is described in further detail with reference to FIGS. 3A-3B.

Accordingly, the outer electrode 204 and the inner electrode 206 may be formed of any material suitable for performing diathermy-involved procedures. The materials may have high electrical conductivity, thermal conductivity, and biocompatibility. For example, the electrodes 204, 206 may comprise materials such as stainless steel, tungsten, platinum and platinum-iridium alloys, gold, silver, and/or nickel alloys.

Note that the electrode assembly 201 shown in FIG. 2A is illustrated in a genericized manner (i.e., without additional components). For example, an electrode assembly typically includes an isolator (a polymer disposed between the outer electrode and the inner electrode); however, the probe here is shown generically for brevity.

FIG. 2B illustrates the distal tip 210 of the probe 108 shown in FIG. 2A, according to certain embodiments. As shown in FIG. 2B, the inner electrode 206 is disposed within and extends through the outer electrode 204. The electrode assembly 201 also includes an isolator (or insulator) 212 disposed between the outer electrode 204 and the inner electrode 206. The isolator 212 also shares the common longitudinal axis (i.e., the longitudinal axis 230) with the electrodes 204, 206 along an entire length thereof. The isolator 212 is configured to prevent electrical current and/or heat from flowing directly between the electrodes 204, 206. The isolator 212 may be comprised of an electrically non-conductive, thermally resistant, biocompatible material such as polytetrafluoroethylene (PTFE), polyether ether ketone (PEEK), or polyimide (PI). Other example materials for the isolator 212 may include polyamide (PA), polypropylene (PP), polyethylene (PE), thermoplastic elastomers (TPE), thermoplastic polyurethane (TPU), or silicones.

The outer electrode 204, the inner electrode 206, and the isolator 212 each have corresponding diameters. The outer electrode 204 has a first diameter 213D that is, for example, a maximum of 0.9 mm (e.g., for a 27 gauge (GA) ophthalmic instrument). The inner electrode 206 has a second diameter 215D that is, for example, a maximum of 0.7 mm. The isolator 212 has a third diameter 217D that is, for example, a maximum of 0.8 mm. In certain embodiments, the diameters 215D and 217D may vary based on the first diameter 213D, which may be between 0.4 mm and 0.9 mm (e.g., for a 20 GA to 27 GA ophthalmic instrument). Based on the first diameter 213D, the diameters 215D and 217D may be determined accordingly to achieve the desired performance such as, e.g., dielectric strength, stiffness, manufacturability, etc.

At the distal tip 210, a first distance (or length) 209L between a distal end 214 of the inner electrode 206 and a distal end 216 of the outer electrode 204 is, for example, at least 0.5 mm. A second distance (or length) 211L between a distal end 218 of the isolator 212 and the distal end 216 of the outer electrode 204 may be, for example, between 0 mm and 1 mm (e.g., 0.5 mm). Accordingly, the first distance 209L is greater than the second distance 211L. In certain embodiments, the distances 209L, 211L may vary based on angles of the distal end 214 and/or a thickness of the isolator 212, which is primarily determined according to a dielectric strength of the isolator 212’s material.

The distal end 214 of the inner electrode 206 includes a beveled surface 224 that is rounded (or curved) to prevent accidental piercing or tearing of tissues while the electrode assembly 201 is inserted in an eye. The beveled surface 224 of the inner electrode 206 and a beveled surface 226 of the isolator 212 are angled relative to the longitudinal axis 230 of the probe 108. For example, the beveled surface 224 of the inner electrode 206 forms an angle 219, and the beveled surface 226 of the isolator 212 forms an angle 221. Both angles 219, 221 are at least 20° (degrees) relative to the longitudinal axis 230. In certain embodiments, the beveled surfaces 224, 226 are grinded to form the angles 219, 221. In some embodiments, one or both of the surfaces 224, 226 may not be beveled, and instead, may be flat surfaces similar to the distal end 216 of the outer electrode 204.

FIG. 3A illustrates a cross-sectional side view of an eye 300 with the probe 108 of FIGS. 2A-2B inserted therein, according to certain embodiments. FIG. 3B illustrates an isometric view of the distal tip 210 of the probe 108 of FIGS. 2A-2B at a treatment area 312 of the eye 300 of FIG. 3A. Accordingly, FIGS. 3A-3B are described together herein for clarity purposes.

The eye 300 includes a vitreous cavity 302 with vitreous 304, a retina 306, and a sclera 308. As shown in FIG. 3A, the probe 108 (e.g., diathermy probe) is inserted into the eye 300 through a cannula 320. The cannula 320 may be used to create an incision through the sclera 308 of the eye 300. In some instances, the cannula 320 may be inserted into an incision made by the surgeon (or other medical professional) using another surgical tool.

The probe 108 is configured to apply heat to a target tissue 310 at the treatment area 312 (e.g., area of operation) within the eye 300. During the ophthalmic procedure, the surgeon may position the probe 108 such that the distal tip 210 is disposed near, but not in contact with, the target tissue 310 at the treatment area 312. In some embodiments, the probe 108 may be movable within the vitreous cavity 302, so that the surgeon can apply heat to other tissues at different treatment areas within the vitreous cavity 302.

When the probe 108 is used to apply heat to target tissue 310 at the treatment area 312, the surgeon is provided with an improved view of the target tissue 310 and/or the treatment area 312 at the distal tip 210 because of the beveled surface 224 of the inner electrode 206, and because the inner electrode 206 is offset from the outer electrode 204. The beveled surface 224 allows the inner electrode 206 to occupy less spatial volume at the distal end 214 in comparison to inner electrodes which have a larger angle (e.g., greater than 30°, 45°, 60°, etc.) and/or broader (or flatter) tip at the distal end 214. Similarly, the outer electrode 204, which occupies greater spatial volume than the inner electrode 206 due to its larger diameter 213D, does not visually block the surgeon’s view of the target tissue 310 and/or the treatment area 312 because the inner electrode 206 extends further distally than the outer electrode 204. Thus, the surgeon has improved visibility of the target tissue 310 and/or the treatment area 312 because the inner electrode 206 and the outer electrode 204 occupy less spatial volume within the eye 300 due to: (1) the beveled surface 224 of the inner electrode 206; and (2) the outer electrode 204 not extending to the distal end 214 of the inner electrode 206 (i.e., the outer electrode 204 does not extend completely over the inner electrode 206).

As a result of the improved visualization of the target tissue 310 and/or the treatment area 312, the surgeon is able to more accurately and efficiently operate inside the eye 300. Accordingly, improving the surgeon’s visibility within the eye 300 helps reduce the likelihood of unwanted and unintentional trauma to ocular tissues, and decreases the duration of the surgery, which further lessens risks for the patient.

At a proximal end of electrode assemblies, electrodes are conductively coupled to sheet metal pins in a connection assembly to relay charges (or electrical current) from the sheet metal pins to the electrodes. In conventional diathermy probes, coupling between the electrodes and the sheet metal pins may involve three or more conductive connections, bent electrodes, and/or an additional bridging wire. As such, a large handpiece may be needed to house such assemblies because they may occupy and/or require a significant amount of space within the handpiece. However, the large handpiece may be uncomfortable for the surgeon to hold during ophthalmic procedures do to the added size and/or weight. Further, such assemblies may also require more time and/or parts to couple the electrodes to the sheet metal pins, which further increases fabrication costs. Consequently, conventional connection assemblies may limit the manufacturing efficiency of diathermy probes. By simplifying the connection assembly as described in FIGS. 4A-4B and 5A-5B, the size requirements of the handpiece, number of components, and manufacturing time to produce such an assembly can be reduced.

FIGS. 4A-4B illustrate cross-sectional views of an example connection assembly 402 that can be used with the probe 108 shown in FIGS. 2A-2B, according to certain embodiments. Accordingly, FIGS. 4A-4B are described together herein for clarity purposes.

As shown in FIG. 4A, the probe 108 includes an internal support 400 configured to hold the electrode assembly 201 within the handpiece 202 of FIG. 2A. In certain embodiments, the internal support 400 is an overmolded stiffener material such as, polycarbonate (PC), polystyrene (PS), acrylonitrile butadiene styrene (ABS), PP, PE, PEEK, PA, or other suitable polymer material. At a distal end 422 of the internal support 400, the internal support 400 includes a stiffening tube 401 that is disposed over the electrode assembly 201 and through an internal base 403. The stiffening tube 401 is at least partially disposed over the outer electrode 204 and is configured to help prevent the outer electrode 204 and/or the inner electrode 206 from cracking or breaking along a length thereof. At a proximal end 420 of the internal support 400, the electrode assembly 201 is conductively coupled to the sheet metal pins 208a-b in the connection assembly 402, for example, as shown in FIG. 4B.

At the connection assembly 402, the first sheet metal pin 208a is conductively coupled to the outer electrode 204 by a first adhesive connection 404a, and the second sheet metal pin 208b is conductively coupled to the inner electrode 206 by a second adhesive connection 404b. Examples of the first adhesive connection 404a and the second adhesive connection 404b (collectively referred to herein as “adhesive connections 404a-b”) include Gernell conductive adhesives, which comprise a polymer matrix and a conductive material that is added to the polymer matrix in different concentrations as particles or powder. The polymer matrix may be an acrylic adhesive, an epoxy adhesive, or a polyurethane adhesive, and the conductive material may be silver, steel, gold, copper, or other conductive materials. Accordingly, the adhesive connections 404a-b may be a silver-based adhesive, steel-based adhesive, gold-based adhesive, copper-based adhesive, etc.

The first adhesive connection 404a is disposed distally relative to the second adhesive connection 404b. In other words, the first adhesive connection 404a is disposed over a distal end 450 of the first sheet metal pin 208a, and the second adhesive connection 404b is disposed between a proximal end 454 and a distal end 456 of the second sheet metal pin 208b. By using the adhesive connections 404a-b, there may be less contaminants than if the sheet metal pins 208a-b were glued or soldered to the electrodes 204, 206.

The adhesive connections 404a-b are configured to relay charges (or electrical currents) received by the sheet metal pins 208a-b to the corresponding electrodes 204, 206. That is, the first adhesive connection 404a relays a charge from the first sheet metal pin 208a to the outer electrode 204, and the second adhesive connection 404b relays another charge from the second sheet metal pin 208b to the inner electrode 206. Charges may be alternatively applied to the first sheet metal pin 208a and the second sheet metal pin 208b to generate heat at the distal tip 210 shown in FIGS. 3A-3B.

Note that FIGS. 4A-4B only illustrate one possible arrangement where the sheet metal pins 208a-b are coupled to the electrodes 204, 206, and that many other arrangements are also within the scope of the disclosure. For example, in certain embodiments, the first sheet metal pin 208a may be coupled to the inner electrode 206 and the second sheet metal pin 208b may be coupled to the outer electrode 204.

FIGS. 5A-5B illustrate cross-sectional views of another example connection assembly 502 that can be used with the probe 108 shown in FIGS. 2A-2B, according to certain embodiments. Accordingly, FIGS. 5A-5B are described together herein for clarity purposes.

As shown in FIG. 5A, the probe 108 includes an internal support 500 configured to hold the electrode assembly 201 within the handpiece 202 of FIG. 2A. At a distal end 522 of the internal support 500, the internal support 500 includes a stiffening tube 501 that is disposed over the electrode assembly 201 and through an internal base 503. At a proximal end 520 of the internal support 500, the electrode assembly 201 is conductively coupled to the sheet metal pins 208a-b in the connection assembly 502, for example, as shown in FIG. 5B.

At the connection assembly 502, the first sheet metal pin 208a is conductively coupled to the outer electrode 204 by a first crimp connection 504a, and the second sheet metal pin 208b is conductively coupled to the inner electrode 206 by a second crimp connection 504b. The first crimp connection 504a is formed by crimping the first sheet metal pin 208a to the outer electrode 204, and the second crimp connection 504b is formed by crimping the second sheet metal pin 208b to the inner electrode 206. The first crimp connection 504a is disposed distally relative to the second crimp connection 504b. In other words, the first crimp connection 504a is formed at a distal end 550 of the first sheet metal pin 208a, and the second crimp connection 504b is formed between a proximal end 554 and a distal end 556 of the second sheet metal pin 208b. By crimping the sheet metal pins 208a-b to the electrodes 204, 206, there may be less contaminants than if the sheet metal pins 208a-b were glued or soldered to the electrodes 204, 206.

The crimp connections 504a-b are configured to relay charges (or electrical current) received by the sheet metal pins 208a-b to the corresponding electrodes 204, 206. That is, the first crimp connection 504a relays a charge from the first sheet metal pin 208a to the outer electrode 204, and the second crimp connection 504b relays another charge from the second sheet metal pin 208b to the inner electrode 206. Charges may be alternatively applied to the first sheet metal pin 208a and the second sheet metal pin 208b to generate heat at the distal tip 210 shown in FIGS. 3A-3B.

The internal support 500 further includes a first plurality of notches 542a, 542b (542a-b) and a second plurality of notches 544a, 544b (544a-b). The first plurality of notches 542a-b align with a first plurality of indentations 546a, 546b (546a-b) on opposing sides of the first sheet metal pin 208a, and the second plurality of notches 544a-b align with a second plurality of indentations 548a,548b (548a-b) on opposing sides of the second sheet metal pin 208b. The notches 542a-b, 544a-b and the indentations 546a-b, 548a-b are configured to help support and/or maintain alignment between the sheet metal pins 208a-b, the electrodes 204, 206, and the crimp connections 504a-b.

Note that FIGS. 5A-5B only illustrate one possible arrangement where the sheet metal pins 208a-b are coupled to the electrodes 204, 206, and that many other arrangements are also within the scope of the disclosure. For example, in certain embodiments, the first sheet metal pin 208a may be coupled to the inner electrode 206 and the second sheet metal pin 208b may be coupled to the outer electrode 204.

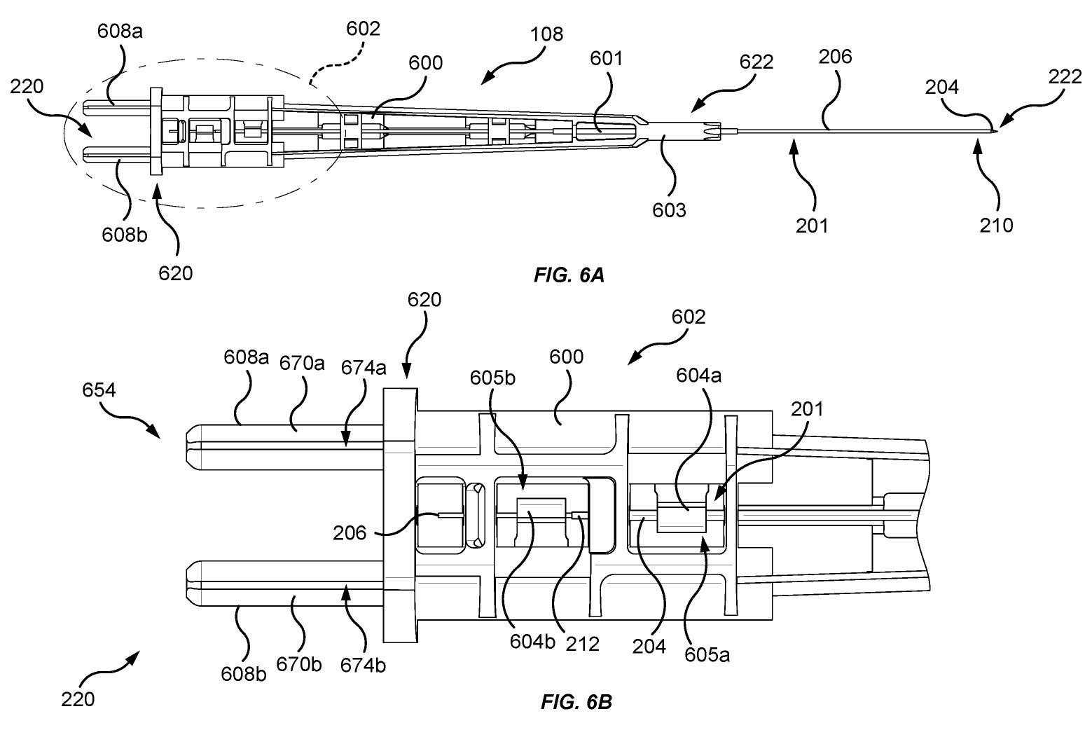

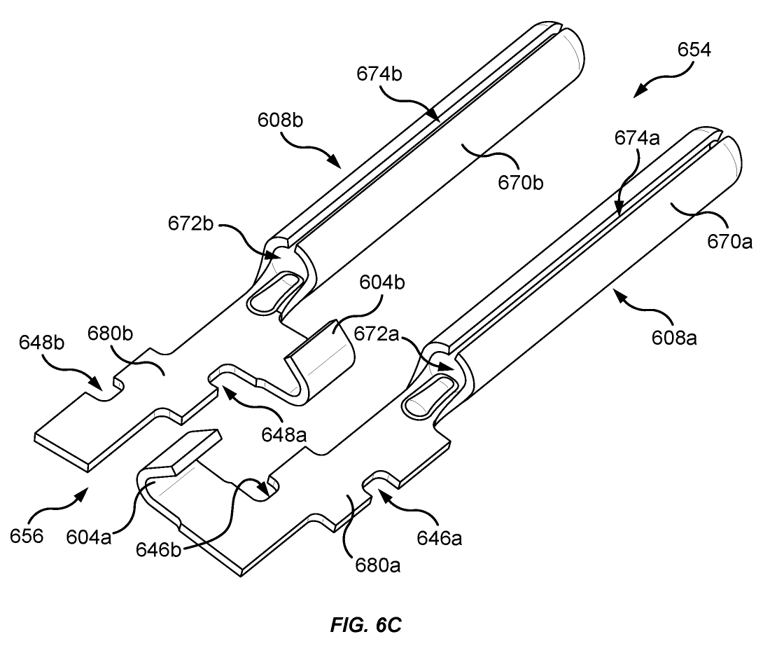

FIGS. 6A-6B show side views of another example connection assembly 602 that can be used with the probe 108 shown in FIGS. 2A-2B, according to certain embodiments. FIG. 6C shows an isometric perspective view of sheet metal pins 608a-b shown FIGS. 6A-6B, according to certain embodiments. Accordingly, FIGS. 6A-6C are described together herein for clarity purposes.

As shown in FIG. 6A, the probe 108 includes an internal support 600 that is overmolded onto a first sheet metal pin 608a and a second sheet metal pin 608b (sheet metal pins 608a-b). The internal support 600 is configured to hold the electrode assembly 201 within the handpiece 202 of FIG. 2A. At a distal end 622 of the internal support 600, the internal support 600 includes a stiffening tube 601 that is disposed over the electrode assembly 201 and through an internal base 603. In certain embodiments, the stiffening tube 601 is overmolded with the internal support 600. At a proximal end 620 of the internal support 600, the electrode assembly 201 is conductively coupled to the sheet metal pins 608a-b in the connection assembly 602, for example, as shown in FIG. 6B.

Similar to the connection assembly 502 of FIGS. 5A-5B, the first sheet metal pin 608a is conductively coupled to the outer electrode 204 by a first crimp connection 604a, and the second sheet metal pin 608b is conductively coupled to the inner electrode 206 by a second crimp connection 604b. In other words, the sheet metal pins 608a-b and the crimp connections 604a-b are formed, and function similar to the sheet metal pins 208a-b and the crimp connections 504a-b described with respect to FIGS. 5A-5B.

However, as shown in FIG. 6C, the first sheet metal pin 608a and the second sheet metal pin 608b further include, respectively, a first curved portion 670a and a second curved portion 670b (curved portions 670a-b) at a proximal end 654 of the sheet metal pins 608a-b. The curved portions 670a-b define a first slit 674a and a second slit 674b (slits 674a-b), as well as a first cavity 672a and a second cavity 672b (cavities 672a-b). The first sheet metal pin 608a and the second sheet metal pin 608b further include a first planar portion 680a and a second planar portion 680b (planar portions 680a-b), respectively, at a distal end 656 of the sheet metal pins 608a-b. The crimp connections 604a-b extend from the planar portions 680a-b. The planar portions 680a-b include a plurality of indentations 646a, 646b (indentations 646a-b) on opposing sides of the first sheet metal pin 608a, and a plurality of indentations 648a, 648b (indentations 648a-b) on opposing sides of the second sheet metal pin 608b.

In view of FIGS. 6A-6C, when the internal support 600 is overmolded onto the sheet metal pins 608a-b, the internal support 600 fills the cavities 672a-b and is formed over the planar portions 680a-b, leaving the crimp connections 604a-b and the curved portions 670a-b exposed. As shown in FIG. 6B, the internal support 600 defines a first aperture 605a and a second aperture 605b (apertures 605a-b) around the crimp connections 604a-b. The apertures 605a-b provide access to the crimp connections 604a-b during assembly of the probe 108. In other words, the internal support 600 is not overmolded over the crimp connections 604a-b, thereby allowing the crimp connections 604a-b to be crimped over the electrodes 204, 206 after the electrodes 204, 206 and the isolator 212 are inserted into the internal support 600.

Overmolding the internal support 600 onto the sheet metal pins 608a-b simplifies the manufacturing process of the probe 108 because the sheet metal pins 608a-b can be held in place within the internal support 600 without the need for additional fixation materials or adhesives. Eliminating adhesives also limits the likelihood of potential contaminants being introduced during assembly of the probe 108. In addition, manufacturing efficiency increases because the crimp connections 604a-b can be crimped to the electrodes 204, 206 after overmolding the internal support 600 (i.e., without having to wait for adhesives to harden).

Further, overmolding the internal support 600 ensures a mechanically robust connection and proper alignment between the sheet metal pins 608a-b and the electrodes 204, 206. Having the internal support 600 fill the cavities 672a-b helps reinforce the alignment and provide improved connectivity (e.g., in comparison to connection assemblies that rely on adhesives) between the sheet metal pins 608a-b and the electrodes 204, 206.

Note that FIGS. 6A-6B only illustrate one possible arrangement where the sheet metal pins 608a-b are coupled to the electrodes 204, 206, and that many other arrangements are also within the scope of the disclosure. For example, in certain embodiments, the first sheet metal pin 608a may be coupled to the inner electrode 206 and the second sheet metal pin 608b may be coupled to the outer electrode 204.

By using one of the connection assemblies 402, 502, or 602, the electrodes 204, 206 are simply and efficiently coupled to the sheet metal pins 208a-b. In other words, the amount of space needed within the handpiece 202 to facilitate the coupling between the electrodes 204, 206 and the sheet metal pins 208a-b is reduced in comparison to connection assemblies which may involve three or more conductive connections, bent electrodes, and/or an additional bridging wire. Thus, the size and/or weight of the handpiece 202 may be reduced. Further, in comparison to conventional connection assemblies, the connection assemblies 402, 502, and 602 require less time and/or parts to couple the electrodes 204, 206 and the sheet metal pins 208a-b, which thereby helps further reduce fabrications costs and improve overall manufacturing efficiency.

The present disclosure may be embodied in other specific forms without departing from its spirit. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: A diathermy probe comprising: a handpiece; and an electrode assembly extending distally from the handpiece, the electrode assembly comprising: an outer electrode; and an inner electrode disposed within and extending through the outer electrode, wherein: the outer electrode and the inner electrode share a common longitudinal axis along an entire length of each of the outer electrode and the inner electrode; and a first sheet metal pin conductively coupled to the outer electrode by a first crimp connection at a proximal end of the handpiece; a second sheet metal pin conductively coupled to the inner electrode by a second crimp connection at the proximal end of the handpiece; and an internal support overmolded onto the first sheet metal pin and the second sheet metal pin.

Embodiment 2: The diathermy probe of Embodiment 1, wherein: the first sheet metal pin comprises: a curved portion defining a cavity therein; and a planar portion extending from the curved portion; and the second sheet metal pin comprises: a curved portion defining a cavity therein; and a planar portion extending from the curved portion.

Embodiment 3: The diathermy probe of Embodiment 2, wherein the internal support is overmolded onto the planar portion of the first sheet metal pin and the planar portion of the second sheet metal pin.

Embodiment 4: The diathermy probe of Embodiment 2, wherein the internal support fills the cavity of the first sheet metal pin and the cavity of the second sheet metal pin.

Embodiment 5: The diathermy probe of Embodiment 1, wherein the internal support defines one or more apertures therethrough that provide access to the first sheet metal pin and the second sheet metal pin.

Embodiment 6: The diathermy probe of Embodiment 1, wherein the internal support aligns the first sheet metal pin and the second sheet metal pin with the inner electrode and the outer electrode.

Embodiment 7: The diathermy probe of Embodiment 1, wherein: the first crimp connection is formed by crimping the first sheet metal pin to the outer electrode; and the second crimp connection is formed by crimping the second sheet metal pin to the inner electrode.

Embodiment 8: The diathermy probe of Embodiment 1, wherein the first sheet metal pin and the second sheet metal pin comprise one or more indentations that align with one or more notches of the handpiece to maintain alignment with the electrode assembly.

Embodiment 9: The diathermy probe of Embodiment 1, wherein: an isolator is disposed between the outer electrode and the inner electrode; and the isolator shares the common longitudinal axis along an entire length of the isolator.

Embodiment 10: The diathermy probe of Embodiment 1, wherein a distal end of the inner electrode comprises a beveled surface that is angled at least 20° (degrees) relative to a longitudinal axis of the diathermy probe.

What is claimed is:

1. A diathermy probe comprising:
a handpiece; and
an electrode assembly extending distally from the handpiece, the electrode assembly comprising:
an outer electrode; and
an inner electrode disposed within and extending through the outer electrode, wherein:
the inner electrode extends further distally from the handpiece than the outer electrode.

2. The diathermy probe of claim 1, wherein a distance between a distal end of the outer electrode and a distal end of the inner electrode is at least 0.5 millimeters (mm).

3. The diathermy probe of claim 1, wherein a distal end of the inner electrode comprises a beveled surface that is angled at least 20° (degrees) relative to a longitudinal axis of the diathermy probe.

4. The diathermy probe of claim 1, wherein the outer electrode has a maximum diameter of 0.9 millimeters (mm).

5. The diathermy probe of claim 1, wherein an isolator is disposed between the outer electrode and the inner electrode.

6. A diathermy probe comprising:
a handpiece; and
an electrode assembly extending distally from the handpiece, the electrode assembly comprising:
an outer electrode; and
an inner electrode disposed within and extending through the outer electrode, wherein:
the outer electrode and the inner electrode share a common longitudinal axis along an entire length of each of the outer electrode and the inner electrode; and
a first sheet metal pin conductively coupled to the outer electrode by a first adhesive connection at a proximal end of the handpiece; and
a second sheet metal pin conductively coupled to the inner electrode by a second adhesive connection at the proximal end of the handpiece.

7. The diathermy probe of claim 6, wherein at least one of the first adhesive connection or the second adhesive connection comprise a polymer matrix and a conductive material.

8. The diathermy probe of claim 6, wherein the first adhesive connection is disposed distally relative to the second adhesive connection.

9. The diathermy probe of claim 6, wherein the first sheet metal pin and the second sheet metal pin comprise one or more indentations that align with one or more notches of the handpiece to maintain alignment with the electrode assembly.

10. The diathermy probe of claim 6, wherein:
an isolator is disposed between the outer electrode and the inner electrode; and
the isolator shares the common longitudinal axis along an entire length of the isolator.

11. A diathermy probe comprising:
a handpiece; and
an electrode assembly extending distally from the handpiece, the electrode assembly comprising:
an outer electrode; and
an inner electrode disposed within and extending through the outer electrode, wherein:
the outer electrode and the inner electrode share a common longitudinal axis along an entire length of each of the outer electrode and the inner electrode; and
a first sheet metal pin conductively coupled to the outer electrode by a first crimp connection at a proximal end of the handpiece; and
a second sheet metal pin conductively coupled to the inner electrode by a second crimp connection at the proximal end of the handpiece.

12. The diathermy probe of claim 11, wherein:
the first crimp connection is formed by crimping the first sheet metal pin to the outer electrode; and
the second crimp connection is formed by crimping the second sheet metal pin to the inner electrode.

13. The diathermy probe of claim 11, wherein:
an isolator is disposed between the outer electrode and the inner electrode; and
the isolator shares the common longitudinal axis along an entire length of the isolator.

14. The diathermy probe of claim 11, wherein the first sheet metal pin and the second sheet metal pin comprise one or more indentations that align with one or more notches of the handpiece to maintain alignment with the electrode assembly.

15. The diathermy probe of claim 11, further comprising:
an internal support that is overmolded onto the first sheet metal pin and the second sheet metal pin.
